(19) United States
(12) Patent Application Publication
(10) Pub. No.: US 2002/0098998 A1
(43) Pub. Date: Jul. 25, 2002

(54) P27 prevents cellular migration

(76) Inventors: Andrew R. Marks, Larchmont, NY (US); Steven O. Marx, New York, NY (US)

Correspondence Address: Cooper & Dunham LLP, 1185 Avenue of the Americas, New York, NY, 10036, US

(21) Appl. No.: 09/766,944

(22) Filed: Jan. 22, 2001

(51) Int. Cl. A61K 31/00; C12Q 1/00; A61K 31/4741

(52) U.S. Cl. 514/1; 514/291; 435/4

(57) ABSTRACT

This invention provides methods of preventing cellular migration and of treating cardiovascular diseases and tumor metastasis by increasing cyclin-dependent kinase inhibitor p27 activity, and methods of identifying chemical compounds for use in such treatments.

The invention disclosed herein was made with Government support under grant numbers RO1HL56180, RO1A139794, and RO3TW00949 from the National Institutes of Health, U.S. Department of Health and Human Services. Accordingly, the U.S. Government has certain rights in this invention.

### BACKGROUND OF THE INVENTION

Throughout this application, various publications are referenced in parentheses by author and year. Full citations for these references may be found at the end of the specification immediately preceding the claims. The disclosures of these publications in their entireties are hereby incorporated by reference into this application to more fully describe the state of the art to which this invention pertains.

Vascular smooth muscle cell (SMC) migration is believed to play a major role in the pathogenesis of many vascular diseases, such as atherosclerosis and restenosis after both percutaneous transluminal angioplasty (PTCA) and coronary stenting (Schwartz, 1997). In normal blood vessels, the majority of SMC reside in the media or middle coat of the vessel, where they are quiescent and possess a “contractile” phenotype, characterized by the abundance of actin- and myosin-containing filaments. In disease states, SMCs migrate from the media to the intima or inner coat of the blood vessel. The process of SMC migration in pathological states involves the synthesis of extracellular matrix, protease enzymes, growth factors such as platelet-derived growth factor (PDGF) and basic fibroblast growth factor (bFGF), and cytokines that further contribute to proliferation and migration (Clowes and Schwartz, 1985; Ferns et al., 1991; Grotendorst et al., 1981; Ihnatowycz et al., 1981; Jawien et al., 1992). Fibroblast growth factor-2 (FGF-2) appears to modulate SMC migration by changing extracellular matrix (ECM)-β1 integrin interactions (Pickering et al., 1997). FGF-2 augments SMC surface expression of α2β1, α3β1 and αvβ1 integrins, thereby resulting in enhanced cellular motility through disassembly of the α-actin stress fiber network (Pickering et al., 1997).

Rapamycin, a macrolide antibiotic, inhibits SMC proliferation both in vitro and in vivo by blocking cell cycle progression at the transition between the first gap (G1) and DNA synthesis (S) phases (Cao et al., 1995; Gallo et al., 1999; Gregory et al., 1993; Marx et al., 1995). The inhibition of cellular proliferation is associated with a marked reduction in cell cycle dependent kinase activity and in retinoblastoma protein phosphorylation in vitro (Marx et al., 1995) and in vivo (Gallo et al., 1999). Down-regulation of the cyclin-dependent kinase inhibitor (CDKI) p27^{kip1} by mitogens is blocked by rapamycin (Kato et al., 1994; Nourse et al., 1994). Pretreatment of rat and human SMC with rapamycin (2 nM) for 48 hours inhibited PDGF-induced SMC migration in a modified Boyden chamber. However, acute rapamycin treatment (6 hours) of rat and human SMC had no effect on migration, suggesting that longer exposure to rapamycin is essential for its anti-migratory actions. In support of these findings, acute 6 hour treatment with rapamycin (1-100 nM), wortmannin and LY294002 of both SMC and Swiss 3T3 cells failed to inhibit PDGF-induced chemotaxis (Higaki et al., 1996). The findings that rapamycin possesses both anti-proliferative and anti-migratory SMC properties led to the suggestion that rapamycin may have important applications in the treatment of disorders such as accelerated arteriopathy that occurs in transplanted hearts and restenosis after percutaneous transluminal angioplasty and placement of coronary stents (Marx et al., 1995; Marx and Marks, 1999; Poon et al., 1996). Rapamycin significantly inhibited neointimal proliferation in a porcine angioplasty model (Gallo et al., 1999) and reversed chronic graft vascular disease in a rodent heart allograft model (Poston et al., 1999). Recent clinical studies have implicated the importance of rapamycin in treating stent restenosis (Sousa et al., 2000).

In p27^{kip1} (−/−) knockout mice, relative rapamycin resistance was demonstrated, and in rapamycin resistant myogenic cells, constitutively low levels of p27^{kip1} were observed, which were not increased with serum withdrawal and rapamycin (Luo et al., 1996). These findings suggested that the ability to block p27^{kip1} down-regulation contributes to the growth inhibitory effects of rapamycin. Transfection of the cyclin-dependent kinase inhibitor p21^{cip1} was shown to inhibit the spreading and attachment of SMC to extracellular matrices and migration in a modified Boyden chamber assay. These findings suggested that p21^{cip1} is probably an adhesion inhibitor, as it prevented the assembly of actin filaments and the translocation of adhesion molecules (Fukui et al., 1997).

The present application discloses that rapamycin has potent inhibitory effects on SMC migration in wild type and p27 (+/−) mice, but not in p27 (−/−) knockout mice, indicating that the cyclin-dependent kinase inhibitor (CDKI) p27^{kip1} plays a critical role in rapamycin's anti-migratory properties and in the signaling pathway(s) that regulates SMC migration.

### SUMMARY OF THE INVENTION

This invention is directed to a method of preventing migration of a cell by increasing intracellular cyclin-dependent kinase inhibitor p27 activity.

The invention provides a method of treating a subject's cardiovascular disease, which comprises administering to the subject a compound which increases intracellular cyclin-dependent kinase inhibitor p27 activity, thereby alleviating the subject's cardiovascular disease.

The invention provides a method of inhibiting tumor metastasis in a subject, which comprises administering to the subject a compound which increases intracellular cyclin-dependent kinase inhibitor p27 activity, thereby inhibiting tumor metastasis.

The invention provides a method of identifying a chemical compound that inhibits cellular migration, which comprises contacting cells whose migration is inhibited when intracellular cyclin-dependent kinase inhibitor p27 activity is increased, or contacting an extract from said cells, with the chemical compound under conditions suitable for increasing p27 activity, and detecting an increase in p27 activity in the presence of the chemical compound so as to thereby identify the chemical compound as a compound which inhibits cellular migration.

The invention provides a method of screening a plurality of chemical compounds not known to inhibit cellular migration to identify a chemical compound which inhibits cellular migration, which comprises:

(a) contacting cells whose migration is inhibited when intracellular cyclin-dependent kinase inhibitor p27 activity is increased, or contacting an extract from said cells, with the plurality of chemical compounds under conditions suitable for increasing p27 activity;

(b) determining if p27 activity is increased in the presence of the plurality of chemical compounds; and if so

(c) separately determining if p27 activity is increased in the presence of each compound included in the plurality of chemical compounds, so as to thereby identify any compound included therein as a compound which inhibits cellular migration.

The invention provides a chemical compound identified by any of the methods described herein.

The invention provides a pharmaceutical composition comprising (a) an amount of a chemical compound identified using any of the methods described herein, or a novel structural and functional homolog or analog thereof, capable of passing through a cell membrane and effective to increase intracellular cyclin-dependent kinase inhibitor p27 activity and (b) a pharmaceutically acceptable carrier capable of passing through the cell membrane.

The invention provides a pharmaceutical composition comprising an amount of a chemical compound identified using any of the methods described herein effective to inhibit cellular migration and a pharmaceutically acceptable carrier.

The invention provides a method for preparing a composition which comprises admixing a carrier and a pharmaceutically effective amount of a chemical compound identified by any of the methods described herein or a novel structural and functional analog or homolog thereof.

The invention provides a method for making a composition of matter which inhibits cellular migration which comprises identifying a chemical compound using any of the methods described herein, and then synthesizing the chemical compound or a novel structural and functional analog or homolog thereof.

The invention provides a method of treating a subject with a cardiovascular disease which comprises administering to the subject a therapeutically effective amount of a chemical compound identified by any of the methods described herein, or a novel structural and functional analog or homolog thereof.

The invention provides a method of inhibiting tumor metastasis in a subject which comprises administering to the subject a therapeutically effective amount of a chemical compound identified by any of the methods described herein, or a novel structural and functional analog or homolog thereof.

The invention provides a use of a chemical compound identified by any of the methods described herein for the preparation of a pharmaceutical composition for treating an abnormality, wherein the abnormality is alleviated by inhibiting cellular migration.

### BRIEF DESCRIPTION OF THE FIGURES

FIGS. 1A-D

Rapamycin potently inhibits migration in smooth muscle cells from wild type, but not p27 (−/−) knockout mice.

(A) Migration of SMCs isolated from wild type mice was determined in the modified Boyden chamber following rapamycin and FK506 treatment. Rapamycin (open bars; 1, 10 and 100 nM) significantly inhibited SMC migration, whereas FK506 demonstrated no effect (blackened bars). *p<0.05 as compared to control. The inset shows an immunoblot demonstrating increased p27^{kip1} levels after rapamycin (100 nM for 48 hours) treatment (lane 2) as compared to untreated proliferating SMC (lane 1).

(B) Migration of SMCs isolated from p27 (−/−) knockout mice was determined in the modified Boyden chamber following rapamycin and FK506 treatment. Only at high concentrations did rapamycin (open bars; 100 and 1000 nM) significantly inhibit SMC migration, whereas FK506 demonstrated no effect (blackened bars). *p<0.05 as compared to control. The inset shows an immunoblot demonstrating the absence of p27^{kip1}.

(C and D) FK506 competes with rapamycin for binding to FKBP12 and inhibits the effects of rapamycin on wild type (C) and p27 (−/−) (D) SMC migration.

FIGS. 2A-B

Lack of effect of rapamycin on murine SMC adhesion.

Wild type (open bars) and p27 (−/−) (blackened bars) SMC were incubated with rapamycin for 48 hours before plating onto either fibronectin (A) or laminin (B) coated plates for 3 hours. The number of adhering cells was determined with a Coulter counter in triplicate and normalized to the number of untreated wild type cells. No significant differences were noted between treated and untreated cells.

FIGS. 3A-C

In vivo administration of rapamycin potently inhibits explant migration of SMC from wild type but not p27 (−/−) knockout animals.

(A) p27 (+/+), p27 (+/−) and p27 (−/−) mice were injected with rapamycin (4 mg/kg/day) for 5 days. The aortas were explanted, and migration of SMC was quantified and is presented as the rapamycin-mediated inhibition of migration as a % of control. Rapamycin significantly inhibited migration in both p27 (+/+) and p27 (+/−) SMC; rapamycin had no effect on p27 (−/−) SMC explant migration

(B) p27 (+/+), p27 (+/−) and p27 (−/−) mice were injected with rapamycin (9 mg/kg/day) for 7 days. Rapamycin inhibited migration in p27 (+/+), p27 (+/−) and p27 (−/−) SMC explants.

(C) p27 (+/+) and p27 (−/−) mice were injected with taxol (20 mg/kg/day) for 7 days. Taxol inhibited migration in p27 (+/+) and p27 (−/−) SMC.

FIG. 4

Impaired migration-inhibitory response to C3 exoenzyme in SMC derived from p27 (−/−) knockout mice.

Migration of SMC isolated from wild type mice (open bars) and p27 (−/−) mice (blackened bars) was determined in the modified Boyden chamber following C3 exoenzyme (2 and 20 μg/ml) treatment for 16 hours. SMC derived from p27 (−/−) mice demonstrated a 25% relative migratory resistance to C3 exoenzyme. *p<0.05 as compared to control.

FIG. 5

Rapamycin and C3 exoenzyme inhibit SMC migration through p27^{kip1}-dependent and -independent pathways.

Rapamycin (Rapa)-FKBP12 inhibits target-of-rapamycin (TOR)-mediated activation/phosphorylation of protein translation modulators 4E-BP1 (a translation initiation factor) and p70 S6 kinase (S6 is a ribosomal protein) (Marx and Marks, 1999) and prevents mitogen-induced down-regulation of p27^{kip1} through an unknown mechanism (dashed lines). Rapamycin inhibits SMC migration through p27^{kip1}-dependent and -independent mechanisms. C3 exoenzyme, which specifically ADP ribosylates and inhibits RhoA, inhibits SMC migration through p27^{kip1}-dependent and-independent (cytoskeleton changes) pathways.

### DETAILED DESCRIPTION OF THE INVENTION

The present invention is directed to a method of preventing migration of a cell by increasing intracellular cyclin-dependent kinase inhibitor p27 activity. In different embodiments of the method, the cell is a smooth muscle cell or a tumor cell.

The invention provides a method of treating a subject's cardiovascular disease, which comprises administering to the subject a compound which increases intracellular cyclin-dependent kinase inhibitor p27 activity, thereby alleviating the subject's cardiovascular disease. In different embodiments, the cardiovascular disease is atherosclerosis, arteriopathy after heart transplantation, or restenosis after angioplasty or coronary stent placement.

The invention provides a method of inhibiting tumor metastasis in a subject, which comprises administering to the subject a compound which increases intracellular cyclin-dependent kinase inhibitor p27 activity, thereby inhibiting tumor metastasis.

In one embodiment of the methods described herein, cyclin-dependent kinase inhibitor p27 activity is increased by increasing C3 exoenzyme activity.

In different embodiments cyclin-dependent kinase inhibitor p27 activity is increased by pharmacological techniques, by recombinant techniques, or by gene therapy. Pharmacological techniques, recombinant techniques, and gene therapy techniques are well known in the art.

The invention provides a method of identifying a chemical compound that inhibits cellular migration, which comprises contacting cells whose migration is inhibited when intracellular cyclin-dependent kinase inhibitor p27 activity is increased, or contacting an extract from said cells, with the chemical compound under conditions suitable for increasing p27 activity, and detecting an increase in p27 activity in the presence of the chemical compound so as to thereby identify the chemical compound as a compound which inhibits cellular migration. In one embodiment, the chemical compound is not previously known to inhibit cellular migration.

The invention provides a method of screening a plurality of chemical compounds not known to inhibit cellular migration to identify a chemical compound which inhibits cellular migration, which comprises:

(a) contacting cells whose migration is inhibited when intracellular cyclin-dependent kinase inhibitor p27 activity is increased, or contacting an extract from said cells, with the plurality of chemical compounds under conditions suitable for increasing p27 activity;

(b) determining if p27 activity is increased in the presence of the plurality of chemical compounds; and if so

(c) separately determining if p27 activity is increased in the presence of each compound included in the plurality of chemical compounds, so as to thereby identify any compound included therein as a compound which inhibits cellular migration.

In different embodiments of the methods described herein, cyclin-dependent kinase inhibitor p27 activity is detected using immunoblots.

In different embodiments of the methods described herein, the cells are smooth muscle cells or tumor cells. In one embodiment, the cells are vertebrate cells. In a further embodiment, the vertebrate cells are mammalian cells. In a still further embodiment, the mammalian cells are human cells.

The invention provides a chemical compound identified by any of the methods described herein.

The invention provides a pharmaceutical composition comprising (a) an amount of a chemical compound identified using any of the methods described herein, or a novel structural and functional homolog or analog thereof, capable of passing through a cell membrane and effective to increase intracellular cyclin-dependent kinase inhibitor p27 activity and (b) a pharmaceutically acceptable carrier capable of passing through the cell membrane.

The invention provides a pharmaceutical composition comprising an amount of a chemical compound identified using any of the methods described herein effective to inhibit cellular migration and a pharmaceutically acceptable carrier.

The invention provides a method for preparing a composition which comprises admixing a carrier and a pharmaceutically effective amount of a chemical compound identified by any of the methods described herein or a novel structural and functional analog or homolog thereof.

The invention provides a method for making a composition of matter which inhibits cellular migration which comprises identifying a chemical compound using any of the methods described herein, and then synthesizing the chemical compound or a novel structural and functional analog or homolog thereof.

The invention provides a method of treating a subject with a cardiovascular disease which comprises administering to the subject a therapeutically effective amount of a chemical compound identified by any of the methods described herein, or a novel structural and functional analog or homolog thereof. In different embodiments, the cardiovascular disease is atherosclerosis, arteriopathy after heart transplantation, or restenosis after angioplasty or coronary stent placement.

The invention provides a method of inhibiting tumor metastasis in a subject which comprises administering to the subject a therapeutically effective amount of a chemical compound identified by any of the methods described herein, or a novel structural and functional analog or homolog thereof.

The invention provides a use of a chemical compound identified by any of the methods described herein for the preparation of a pharmaceutical composition for treating an abnormality, wherein the abnormality is alleviated by inhibiting cellular migration. In different embodiments, the abnormality is a cardiovascular disease or a tumor metastasis. In different embodiments, the cardiovascular disease is atherosclerosis, arteriopathy after heart transplantation, or restenosis after angioplasty or coronary stent placement.

In the subject invention, a “pharmaceutically effective amount” is any amount of a compound which, when administered to a subject suffering from a disease against which the compound is effective, causes reduction, remission, or regression of the disease. Furthermore, as used herein, the phrase “pharmaceutically acceptable carrier” means any of the standard pharmaceutically acceptable carriers. Examples include, but are not limited to, phosphate buffered saline, physiological saline, water, and emulsions, such as oil/water emulsions.

A “structural and functional analog” of a chemical compound has a structure similar to that of the compound but differing from it in respect to a certain component or components. A “structural and functional homolog” of a chemical compound is one of a series of compounds each of which is formed from the one before it by the addition of a constant element. The term “analog” is broader than and encompasses the term “homolog”.

This invention will be better understood from the Experimental Details which follow. However, one skilled in the art will readily appreciate that the specific methods and results discussed are merely illustrative of the invention as described more fully in the claims which follow thereafter.

### Experimental Details

### Materials And Methods

Reagents: Dulbecco Modified Eagle Medium (DMEM) and trypsin were obtained from GIBCO (Grand Island, N.Y.), recombinant bFGF was obtained from Biosource International (Camarillo, Calif.), and paclitaxel was obtained from Sigma (St. Louis, Mo.). Rapamycin was a gift from Dr. Suren Sehgal (Wyeth-Ayerst Laboratories, Princeton, N.J.)

Expression of C3 exoenzyme: C3 exoenzyme was prepared as previously described (Dillon and Feig, 1995). The Glutathione S Transferase (GST)-C3 exoenzyme cDNA (gift of Dr. Judy Meinkoth, University of Pennsylvania) was transformed into competent BL21. Protein expression was induced with 200 μM isopropylthiogalactoside (IPTG) at 32° C. for 3 hours. Lysates were prepared and incubated with GST-sepharose beads for 1 hour at 4° C. The beads were washed and incubated overnight at 4° C. with 3 units/ml thrombin (for cleavage of the C3 exoenzyme from the GST fusion protein), which was removed by incubating the supernatant with antithrombin-sepharose beads for 1 hour at 4° C. The supernatant was concentrated with a Centricon-10 (Amicon Inc, Beverly, Mass.). Protein concentration was determined by Bradford assay and the supernatant was aliquoted and frozen in liquid nitrogen. The samples were run on SDS-PAGE and stained with Coomassie to conform correct expression of the GST fusion protein and cleavage/purification of C3 exoenzyme before use (Seasholtz et al., 1999).

Cell Culture: The murine aortic SMCs were obtained from the explant migration experiments described below, and were subcultured in DMEM containing 20% fetal bovine serum (FBS) at 37° C. in a humidified 95% air-5% CO₂ atmosphere (Kobayashi et al., 1993). The growth medium was changed every other day until 80% confluence was reached. The cells used for experiments were from passages #3-6. Verification of SMC phenotype was determined by positive fluorescent staining for α-actin and negative staining for Factor VIII antigen. Cell viability was 95% or greater as determined by trypan blue exclusion at the conclusion of each experiment.

SMC Adhesion Assay: The adhesion assay was performed as previously described (Wang et al., 1997). Murine SMCs were treated with rapamycin or vehicle for 48 hours. SMCs (5×10⁵/ml in DMEM supplemented with 0.2% bovine serum albumin (BSA)) were loaded onto 12-well plates pre-coated with laminin or fibronectin. After 3 hours, the media containing nonadherent cells were removed, and cell numbers were determined by triplicate counts using a Coulter Counter (Model Z1, Coulter Electronics, Beds, England).

SMC migration assay: Migration was measured using a 48 well modified Boyden chamber housing a polycarbonate filter with 8 μm pores as described previously (Bornfeldt et al., 1994; Poon et al., 1996). Each membrane was coated with 0.1 mg/ml of collagen in 0.2 M acetic acid for 24 hours before each assay. For each assay, 50 ng/ml of bFGF in DMEM was loaded in quadruplicate wells in the bottom chamber. BSA (0.2% in DMEM without bFGF) was used as a negative control. Rapamycin, FK506 or C3 exoenzyme was directly added to the growth medium for either 48 hours (rapamycin and FK506) or 16 hours (C3 exoenzyme) before the cells were trypsinized, and counted with a hemacytometer. An equal number of cells (2×10⁵/ml) in 50 μl was loaded to the top chamber of each well. After 6 hours, non-migrating cells were scraped from the upper surface of the filter. Cells on the lower surface were fixed with methanol and stained with Giemsa stain (Fisher Scientific, N.Y.). The number of SMC on the lower surface of the filter was determined by counting four high power (×200) fields of constant area per well. Values are expressed as the percentage of cells migrating in response to bFGF after subtraction of the negative control (DMEM+BSA). Experiments were performed at least twice using quadruplicate wells.

Aortic SMC explant migration: Wild type C57BL/6 mice were purchased from Jackson Laboratory (Bar Harbor, Me.). The p27 (+/−) and p27 (−/−) knockout mice were kindly provided by Dr. Andrew Koff of Memorial Sloan-Kettering Cancer Institute (Kiyokawa et al., 1996). The mice received one of three different treatment protocols (9 mg/kg/day for 7 days, 4 mg/kg/day for 5 days, or 2 mg/kg/day for 2 days) of rapamycin via intraperitoneal (IP) injection. The control group was treated with vehicle alone (0.2% sodium CMC, polysorbate 0.25%; St. Louis, Mo.). At the conclusion of the treatment protocol, the mice were euthanized with 100 mg/kg of pentobarbital, the aortas excised and the adventitia and surrounding connective tissue were removed. The aortas were then opened by a longitudinal cut and the intima, as well as a thin portion of the subjacent media, were removed. The media were divided into 2 mm×2 mm pieces and placed in 6 well tissue culture plates (35 mm, 22.6 mm diameter, Costar, Cambridge, Mass.) containing DMEM with 20% FBS. The culture media was changed every other day. The migration of SMC out of the explant was observed under the microscope daily following explant. The total number of cells explanted was determined for each animal's explants on a daily basis. The results in FIG. 5 are presented as the mean percentage (±SD) of inhibition of migration (by rapamycin or taxol) as compared to control (untreated) for at least 4 animals from each group. The SMC phenotype was confirmed as previously described (Spector et al., 1997).

Immunoblots: Immunoblots were prepared using procedures previously described in Luo et al. (1996) SMC growing in log phase or treated with rapamycin (100 nM for 48 hours) were washed twice with ice cold phosphate buffered saline (PBS) and lysates prepared using a modified RIPA buffer as previously described (Poon et al., 1996). Lysates were clarified by centrifugation for 20 minutes at 14,000 rpm at 4° C. Protein concentrations were determined by Bradford assay with BSA as a standard (Bradford, 1976). Protein extracts (30 μg) were size-fractionated on SDS-12% polyacrylamide gels and transferred to nitrocellulose. Filters were blocked with PBS-0.1% Tween 20 and 5% dry milk for 1 hour at room temperature, followed by incubation with a mouse monoclonal P27^{kip1} antibody (F8 antibody, Santa Cruz Biotechnology Inc, Santa Cruz, Calif.) for 2 hours. Filters were washed with PBS-0.1% Tween 20 and then incubated with a secondary antibody conjugated to peroxidase for 1 hour. Filters were washed with PBS-0.1% Tween 20; signals were detected using chemiluminescence detection system (ECL) followed by exposure to Kodak XAR film.

Statistics: Data are presented as the mean ± standard deviation (SD) of the independent experiments. Statistical significance was determined by one way analysis of variance (ANOVA) and Fisher's PLSD test (StatView 4.01; Brain Power, Inc., Calabasas, Calif.). A paired t test (StatView 4.01) was used to analyze all data. A p value of <0.05 was considered statistically significant.

### Results

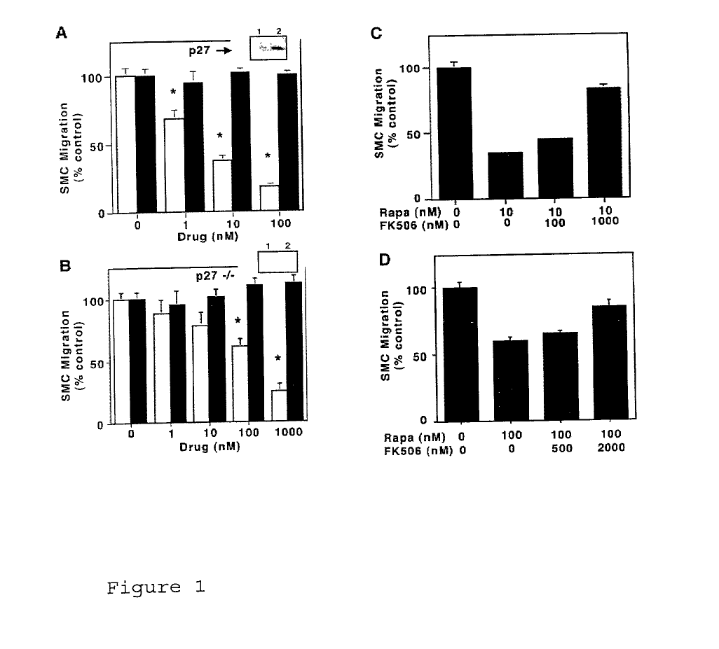

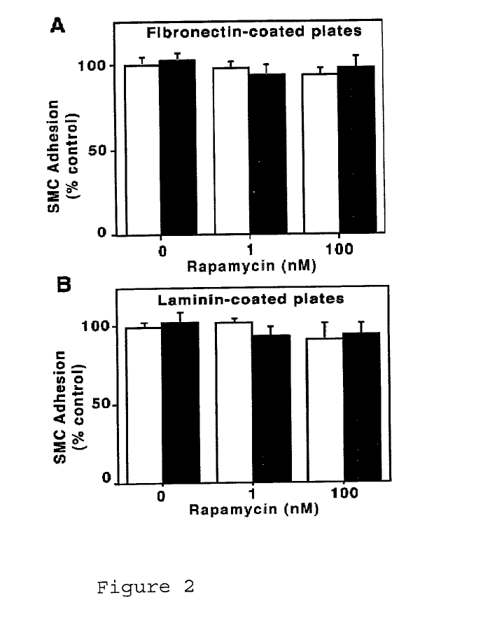

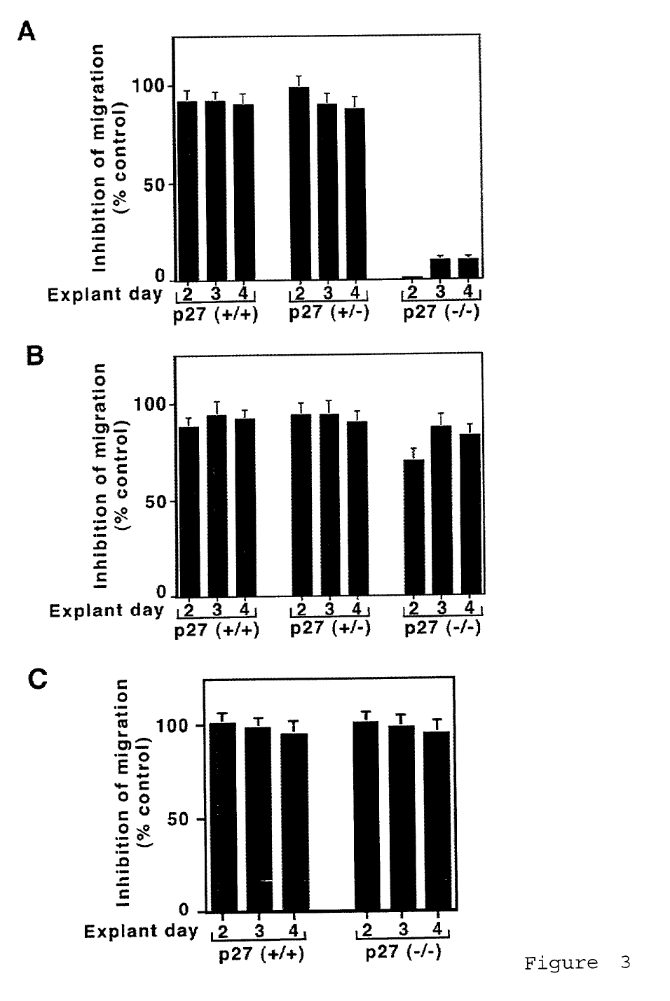

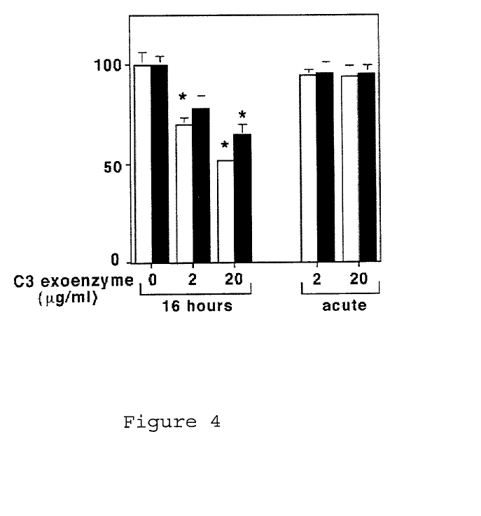

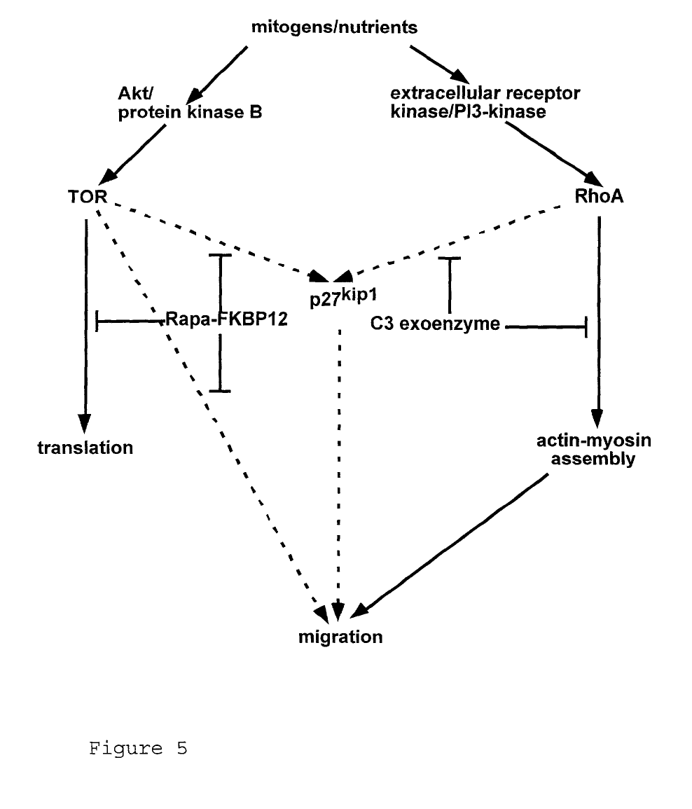

The inhibitory effects of rapamycin on the migration of SMCs isolated from wild type and p27 (−/−) knockout mice were determined. In wild type murine SMC, rapamycin treatment for 48 hours demonstrated a significant inhibitory effect on bFGF-induced SMC migration (FIG. 1A, open bars). The inhibition was concentration dependent between 1 nM and 100 nM, with an IC₅₀ of ˜2 nM. In contrast, no significant inhibition of migration by rapamycin (1 nM to 10 nM) was observed in the p27 (−/−) SMC (FIG. 1B, open bars). At higher concentrations (100 nM), an approximately 35% inhibition was observed; the IC₅₀ in p27 (−/−) cells was ˜200 nM, representing a 100 fold increased IC₅₀ as compared to wild type SMC. Addition of rapamycin to either the upper or lower chambers immediately prior to incubation had no effect on SMC migration. FK506, an agent that binds to the same cytosolic receptor (FKBP12) as rapamycin, had no effect on murine SMC migration (FIGS. 1A and 1B, blackened bars). The inhibition of migration of wild type murine SMC by rapamycin (10 nM) was competitively inhibited by a 100-fold molar excess of FK506 (FIG. 1C). The rapamycin-induced inhibition of migration (100 nM) in the p27 (−/−) SMC was also competitively inhibited by a 20 fold molar excess of FK506 (FIG. 1D). These data indicate that the inhibition of migration was mediated through rapamycin's binding to FKBP12. Treatment of wild type murine SMC with rapamycin (100 nM for 48 hours) caused a significant increase in p27^{kip1} protein levels (FIG. 1A, inset); in contrast, no p27^{kip1} was detected in p27 (−/−) SMC (FIG. 1B, inset). Although rapamycin inhibits SMC proliferation, the differences in migration do not reflect proliferation as equal numbers of cells were loaded into the Boyden chamber. To confirm this, the numbers of cells in the upper and lower chambers after the 6 hour incubation were equal in the untreated and treated wild type and p27 (−/−) SMC. In addition, no differences in cell viability were noted between untreated and rapamycin treated SMC obtained from wild type and p27 (−/−) animals. No morphologic differences were observed between untreated and rapamycin (100 nM for 48 hours) treated SMC isolated from wild type mice and p27 (−/−) mice.

Since migration is dependent upon the adhesion of the SMC to the Boyden chamber membrane, adhesion assays were performed using fibronectin and laminin-coated plates. SMC obtained from p27 (−/−) animals demonstrated no differences in adhesion as compared to SMC obtained from wild type animals on both fibronectin and laminin coated plates. Furthermore, rapamycin treatment (100 nM for 48 hours) did not affect cell adhesion in either wild type or p27 (−/−) SMC (FIG. 2).

To assess the in vivo effects of rapamycin on SMC migration in the p27 (−/−) animals, the ability of SMC to migrate out of the murine aortic explants and establish cell cultures was examined. Rapamycin was not added to the culture medium after the aortas were explanted. Explant migration of aortic SMC was performed using wild type C57BL/6, p27 (+/−), or p27 (−/−) mice. SMC from wild type, p27 (+/−) and p27 (−/−) migrated out of the aortic explant by day #2. In animals treated with rapamycin (4 mg/kg/day for 5 days), ˜85% inhibition of migration as compared to untreated animals was observed in the wild type and p27 (+/−) groups (p<0.05). In contrast, no rapamycin-mediated inhibition of migration was observed in p27 (−/−) group (p<0.05, FIG. 3A), indicating that p27^{kip1} plays a critical role in the rapamycin-mediated inhibition of SMC migration. At higher doses (9 mg/kg/day for 7 days), equivalent levels of rapamycin-mediated inhibition of migration were observed in wild type, p27 (+/−) and p27 (−/−) cells (FIG. 3B). At lower doses (2 mg/kg/day for 2 days), no rapamycin-mediated inhibition of migration was observed. These results are consistent with the findings obtained in the modified Boyden chamber for p27 (−/−) cells and suggests the presence of both p27^{kip1}-dependent and p27^{kip1}-independent pathways mediating rapamycin's SMC anti-migratory actions. In order to demonstrate that agents that did not perturb the p27^{kip1} pathway could inhibit migration in p27 (−/−) animals, wild type and p27 (−/−) animals were treated with taxol (20 mg/kg/day for 7 days) (Sollott et al., 1995). No differences in taxol-mediated inhibition were observed in the two groups (FIG. 3C).

Recent data suggests that the Ras/RhoA mitogenic pathway regulates the destruction of p27^{kip1}. C3 exoenzyme, which adenosine diphosphate (ADP)-ribosylates and inactivates RhoA, inhibited PDGF-induced p27^{kip1} degradation. These findings suggest that activation of RhoA by mitogens is necessary for degradation of p27^{kip1} (Weber et al., 1997). In addition, thrombin-induced vascular SMC DNA synthesis and migration were inhibited by C3 exoenzyme (Seasholtz et al., 1999). We sought to determine whether this inhibition of migration was mediated, in part, by regulating p27^{kip1} levels. SMC from wild type and p27 (−/−) animals were exposed to either 2 μg/ml or 20 μg/ml C3 exoenzyme for 16 hours, trypsinized and loaded into the upper chamber of the Boyden chamber. C3 exoenzyme significantly inhibited bFGF-mediated SMC migration in wild type cells (FIG. 4, open bars). SMC from p27 (−/−) animals demonstrated a 25% relative resistance to C3 exoenzyme (FIG. 4, blackened bars). SMC that were acutely exposed to C3 exoenzyme demonstrated no inhibition of migration. These results implicate p27^{kip1} as a regulator, in part, of both rapamycin and C3 exoenzyme-mediated inhibition of SMC migration.

### Discussion

Rapamycin has been shown previously to inhibit rat, porcine, and human SMC migration (Poon et al., 1996). In addition, rapamycin reduces intimal thickening by 50% after coronary angioplasty in the porcine model (Gallo et al., 1999). The rapamycin anti-restenotic effect is characterized by an inhibition of the SMC response to coronary injury with a concomitant decrease in retinoblastoma protein (pRb) phosphorylation as well as an increase in p27^{kip1} levels, thereby resulting in cell-cycle arrest (Gallo et al., 1999; Marx et al., 1995). The cyclin-dependent kinase inhibitor (CDKI) p27^{kip1} inhibits the regulatory activities of cyclin/CDK complexes including cyclinE/CDK2 by directly binding to them and, in turn, blocking the phosphorylation of retinoblastoma protein (pRb) (Kato et al., 1994; Nourse et al., 1994). Thus, p27^{Kip1} is a regulator of cell proliferation; reduction of p27^{kip1} protein levels during the late G₁ phase is required for cyclin/CDK complex activation and cell cycle progression in certain cell lines. The CDKI p27^{kip1} is present at high levels in quiescent cells and upon mitogenic stimulation is downregulated (Kato et al., 1994; Nourse et al., 1994). Down-regulation of p27^{kip1} by mitogens can be blocked by the immunosuppressant rapamycin (Nourse et al., 1994).

The function of p27^{Kip1} is clinically relevant because of the connections that have been made between the down-regulation and enhanced degradation of p27^{Kip1} in colorectal, stomach, breast, and small-cell lung cancers (Steeg and Abrams, 1997). Furthermore, the regulation of the CDKI p27^{kip1} plays a critical role in the regulation of SMC proliferation in vivo. Decreased levels of p27^{kip1} in the vessel wall has been associated with increased neointimal response after percutaneous transluminal angioplasty (PTCA) (Braun-Dullaeus and al., 1997; Tanner et al., 1998). Angiotensin II stimulation of quiescent vascular SMC in which P27^{kip1} levels are high results in SMC hypertrophy but induces SMC hyperplasia when levels of p27^{kip1} are low as occurs in the presence of mitogens (Braun-Dullaeus et al., 1999). The findings disclosed in the present application suggest that agents that increase p27^{kip1} levels in vivo may have both an anti-proliferative and anti-migratory effect.

Although the regulation of p27^{kip1} can occur at the mRNA level (Hengst and Reed, 1996), most studies have supported the concept that p27^{kip1} is regulated post-transcriptionally and involves ubiquin (Ub)-proteasome dependent degradation (Pagano et al., 1995). Targeting of p27^{kip1} for ubiquitin is believed to involve phosphorylation of p27^{kip1} by cyclin E-cdk2 complex (Sheaff et al., 1997; Vlach et al., 1997). Recently, a ubiquin-proteasome independent pathway has been described that involves proteolytic processing that rapidly clips off the cyclin-binding domain. This ubiquitin independent processing is ATP-dependent and sensitive to proteasome-specific and chymotrypsin inhibitors (Shirane et al., 1999).

In addition, p27^{kip1} levels have been shown to be regulated by the Ras/RhoA mitogenic pathway. Overexpression of a dominant negative Ras or RhoA inhibited the platelet derived growth factor (PDGF) induced degradation of p27^{kip1} C3 exoenzyme, which ADP-ribosylates and inactivates RhoA, inhibited PDGF-induced p27^{kip1} degradation (Hirai et al., 1997; Weber et al., 1997) and inhibited thrombin-mediated vascular SMC proliferation and migration (Seasholtz et al., 1999). In Swiss 3T3 fibroblasts, it has been shown that Rho can be activated by extracellular ligands (lysophosphatidic acid) and that Rho activation can lead to the assembly of contractile actin-myosin filaments and focal adhesion complexes (Hall, 1998). Rac, a member of the Rho subfamily, has been shown to induce actin-rich surface protrusions (filopodia); Rac can activate Rho (although in fibroblasts this is interaction is weak and delayed) (Hall, 1998). Generation of phosphatidylinositol-3,4,5-trisphosphate (PIP3) by PI 3-kinase activity is essential for receptor-mediated activation by Rac in mammalian cells and a PI3 kinase homolog, TOR2 (target of rapamycin 2) controls Rholp activation in Saccharomyces cerevisiae (Hall, 1998; Schmidt et al., 1997). These observations suggests that the Rho GTPase family is one of the key regulatory molecules that link surface receptors to the organization of the actin cytoskeleton. Rapamycin has not been shown to interact with the Rho GTPase family, although it is interesting that inhibition of both Rho (Hirai et al., 1997; Weber et al., 1997) and mTOR (Brown et al., 1994; Nourse et al., 1994; Sabatini et al., 1994) are both associated with increased levels of the CDKI, p27^{kip1}.

The extracellular matrix (ECM) plays an essential role in the regulation of cell proliferation. Human capillary endothelial cells that were prevented from spreading (either mechanically or pharmacologically with cytochalasin or actomyosin) exhibited normal activation of mitogen-activated kinases, but failed to progress through G1 phase (Huang et al., 1998). This shape dependent block in the cell cycle was correlated with a failure to down-regulate p27^{kip1}, up-regulate cyclin D1 and phosphorylate pRb (Huang et al., 1998). Therefore, the accumulation of p27^{kip1} in cells prevented from spreading suggests that p27^{kip1} could play a role in the shape-dependent cell cycle arrest produced by cell rounding. Signaling pathway components that could be responsible for transducing the accumulation of p27^{kip1} include Rho, which is involved in integrin-mediated changes in the cytoskeleton tension and shape, and the integrin-linked kinase, which has been shown to reduce the inhibitory actions of p27^{kip1} and to promote anchorage-independent growth (Chrzanowska-Wodnicka and Burridge, 1996; Hotchin and Hall, 1995; Huang et al., 1998; Radeva et al., 1997).

The p21 CDKI (Cip1) has been shown to inhibit SMC migration in vitro (Fukui et al., 1997; Witzenbichler et al., 1999). The spreading and attachment of the p21^{Cip1} transfected rabbit aortic SMC to extracellular matrices (ECM) were inhibited compared to that of control vector-transfected cells. Cip1 transfected SMC maintained a round conformation on fibronectin. Moreover, p21^{Cip1} transfected SMC demonstrated significantly reduced PDGF-BB mediated migration in a modified Boyden chamber (with fibronectin coated membranes). Therefore, p21^{cip1} probably acts as an adhesion inhibitor, since it prevents the assembly of actin filaments and the translocation of adhesion molecules (Fukui et al., 1997). Interestingly, our study indicates that induction of p27^{kip1} with rapamycin did not affect adhesion to collagen of either wild type or p27 (−/−) cells.

The homeobox transcription factor Gax is expressed in quiescent vascular SMC and is down-regulated during SMC proliferation and vascular injury (Witzenbichler et al., 1999). Gax up-regulates p21^{cip1} and inhibits vascular SMC proliferation and migration (Witzenbichler et al., 1999). p21^{cip1} mediates the growth inhibitory actions of Gax; overexpression of Gax does not have anti-proliferative or anti-migratory effects in cells derived from p21 (−/−) mice (Smith et al., 1997; Witzenbichler et al., 1999). Gax was unable to inhibit the migration of fibroblasts which lacked p21^{cip1} (Witzenbichler et al., 1999). Transfection of a Gax cDNA inhibited PDGF-, bFGF-, and hepatocyte growth factor-induced vascular SMC migration (Witzenbichler et al., 1999). Cell cycle arrest by either p16 or p21 is essential for Gax-induced inhibition of migration. Interestingly, overexpression of Gax cDNA, which increases p21^{cip1}, had no effect on the adhesion of cells to collagen and vitronectin coated plates. Therefore, in contrast to the fibronection adhesion defect shown in cells transfected with p21^{cip1}, cells transfected with Gax cDNA demonstrated no collagen/vitronectin adhesion defect. However, the studies reported conflicting information regarding the effects of overexpression of p21^{cip1} on SMC migration; p21^{cip1} transfection of rabbit vascular SMC inhibited migration in a fibronectin coated Boyden chamber (Fukui et al., 1997), whereas p21^{cip1} transfection in rat vascular SMC had no effect in a collagen/vitronectin Boyden chamber (Witzenbichler et al., 1999).

In conclusion, rapamysin and C3 exoenzyme inhibit smooth muscle cell migration through p27^{kip1}-dependent and independent pathways (FIG. 5). This intriguing finding implicates p27^{kip1} in the signaling pathway(s) that regulate both SMC proliferation and migration. Technologies (e.g., pharmacologic, recombinant and/or gene therapy) aimed at increasing p27^{kip1} are expected to have dramatic effects on the amelioration of restenosis after angioplasty or stent placement, or on accelerated arteriopathy after cardiac transplantation, as well as in cancer therapy where cellular migration is a key element in tumor metastasis.

### References

Bornfeldt, K. E., Raines, E. W., Nakano, T., Graves, L. M., Krebs, E. G., and Ross, R. (1994). Insulin-like growth factor-I and platelet-derived growth factor-BB induce directed migration of human arterial smooth muscle cells via signaling pathways that are distinct from those of proliferation. J Clin Invest 93, 1266-1274.

Bradford, M. M. (1976). A rapid and sensitive method for the quantitation of microgram quantitites of protein utilising the principle of protein-dye binding. Anal Biochem 72, 248-254.

Braun-Dullaeus, R. C., and al., e. (1997). Loss of p27kip1 and induction of Cdk1 in the rat carotid artery following balloon catheter injury. In vivo and in vitro influence of rapamycin. FASEB J 11, A153 (abstract).

Braun-Dullaeus, R. C., Mann, M. J., Ziegler, A., von der Leyen, H. E., and Dzau, V. J. (1999). A novel role for the cyclin-dependent kinase inhibitor p27kip1 in angiotension II-stimulated vascular smooth muscle cell hypertrophy. J. Clin. Invest. 104, 815-823.

Brown, E., Albers, T., Shin, T., Ichikawa, K., Keith, C., Lane, W., and Schreiber, S. (1994). A mammalian protein targeted by G1-arresting rapamycin complex. Nature (London) 369, 756-758.

Cao, W., Mohacsi, P., Shorthouse, R., Pratt, R., and Morris, R. (1995). Effects of rapamycin on growth factor-stimulated vascular smooth muscle cell DNA synthesis: inhibition of basic fibroblast growth factor and platelet-derived growth factor action and antagonism of rapamycin by FK506. Transplantation 59, 390-395.

Chrzanowska-Wodnicka, M., and Burridge, K. (1996). Rho-stimulated contractility drives the formation of stress fibers and focal adhesions. J. Cell Biol. 133, 1403-1415.

Clowes, A., and Schwartz, S. M. (1985). Significance of quiescent smooth muscle migration in the injured rat carotid artery. Circ Res. 56, 139-45.

Dillon, S. T., and Feig, L. A. (1995). Purification and assay of recombinant C3 transferase. Methods in Enzymology 256, 174-184.

Ferns, G. A. A., Raines, E. W., Sprugel, H., Motani, A. S., Reidy, M. A., and Ross, R. (1991). Inhibition of neointimal smooth muscle accumulation after angioplasty by an antibody to PDGF. Science 253, 1129-1132.

Fukui, R., Shibata, N., Kohbayashi, E., Amakawa, M., Furutama, D., Hoshiga, M., Negoro, N., Nakakouji, T., Ii, M., Ishihara, T., and Ohsawa, N. (1997). Inhibition of smooth muscle cell migration by the p21 cyclin-dependent kinase inhibitor (Cip1). Atherosclerosis 132, 53-59.

Gallo, R., Padurean, A., Jayaraman, T., Marx, S. O., Roque, M., Adelman, S., Chesebro, J., Fallon, J., Fuster, V., Marks, A. R., and Badimon, J. J. (1999). Inhibition of intimal thickening after balloon angioplasty in porcine coronary arteries by targeting regulators of the cell cycle. Circulation 99, 2164-2170.

Gregory, C., Huie, P., Billingham, M., and Morris, R. (1993). Rapamycin inhibits arterial intimal thickening caused by both alloimmune and mechanical injury. Transplantation 55, 1409-1418.

Grotendorst, G. R., Seppa, H. E. J., Kleinman, H. K., and Martin, G. R. (1981). Attachment of smooth muscle cells to collagen and their migration toward platelet derived growth factor. Proc Natl Acad Sci USA 78, 3669-3672.

Hall, A. (1998). Rho GTPases and the actin cytoskeleton. Science 279, 509-514.

Hengst, L., and Reed, S. I. (1996). Translational control of p27^{kip1} accumulation during the cell cycle. Science 271, 1861-1864.

Higaki, M., Sakaue, H., Ogawa, W., Kasuga, M., and Shimokado, K. (1996). Phosphatidylinositol 3-kinase-independent signal transduction pathway for platelet-derived growth factor-induced chemotaxis. J. Biol. Chem. 271, 29342-29346.

Hirai, A., Nakamura, S., Noguchi, Y., Yasuda, T., Kitagawa, M., Tatsuno, I., Oeda, T., Tahara, K., Terano, T., Narumiya, S., Kohn, L. D., and Saito, Y. (1997). Geranylgeranylated rho small GTPase(s) are essential for the degradation of p27kip1 and facilitate the progression from G1 to S phase in growth-stimulated rat FRTL-5 cells. J. Biol. Chem. 272, 13-16.

Hotchin, N. A., and Hall, A. (1995). The assembly of integrin adhesion complexes requires both extracellular matrix and intracellular rho/rac GTPases. J. Cell. Biol. 131, 1857-65.

Huang, S., Chen, C. S., and Ingber, D. E. (1998). Control of cyclin D1, p27kip1 and cell cycle progression in human capillary endothelial cells by cell shape and cytoskeletal tension. Mol. Biol. Cell 9, 3179-3193.

Ihnatowycz, I. O., Winocour, P. D., and Moore, S. (1981). A platelet-derived factor chemotatic for rabbit smooth muscle cells in culture. Artery 9, 316-317.

Jawien, A., Bowen-Pope, D. F., Lindner, V., Schwartz, S. M., and Clowes, A. W. (1992). Platelet-derived growth factor promotes smooth muscle migration and intimal thickening in a rat model of balloon angioplasty. J Clin Invest. 89, 507-511.

Kato, J. M., Matsuoka, M., Polyak, K., Massague, J., and Sherr, C. J. (1994). Cyclic AMP-induced G1 phase arrest mediated by an inhibitor (p27kip1) of cyclin-dependent kinase-4 activation. Cell 79, 487-496.

Kiyokawa, H., Kineman, R. D., Manova-Todorova, K. O., Soares, V. C., Hoffman, E. S., Ono, M., Khanam, D., Hayday, A. C., Frohman, L. A., and Koff, A. (1996). Enhanced growth of mice lacking the cyclin-dependent kinase inhibitor function of p27^{kip1}. Cell 85, 721-732.

Kobayashi, S., Mimura, Y., Naitoh, T., Kimura, I., and Kimura, M. (1993). Chemical structure-activity of cnidium rhizone-derived phthalides for the competence inhibition of proliferation in primary culture of mouse aorta smooth muscle cells. Japan J. Pharmacol 63, 353-359.

Luo, Y., Marx, S. O., Kiyokawa, H., Koff, A., Massague, J., and Marks, A. R. (1996). Rapamycin resistance tied to defective regulation of p27^{kip1}. Mol. Cell. Biol. 16, 6744-6751.

Marx, S. O., Jayaraman, T., Go, L. O., and Marks, A. R. (1995). Rapamycin-FKBP inhibits cell cycle regulators of proliferation in vascular smooth muscle cells. Circ Res 76, 412-417.

Marx, S. O., and Marks, A. R. (1999). Cell cycle progression and proliferation despite 4BP-1 dephosphorylation. Mol Cell Biol. 19, 6041-6047.

Nourse, J., Firpo, E., Flanagan, W. M., Coats, S., Polyak, K., Lee, M., Massague, J., Crabtree, G., and Roberts, J. M. (1994). Interleukin-2-mediated elimination of the p27kip1 cyclin-dependent kinase inhibitor prevented by rapamycin. Nature (London) 372, 570-573.

Pagano, M., Tam, S. W., Theodoras, A. M., Beer-Romero, P., G., D. S., Chau, V., Yew, P. R., Draetta, G. F., and Rolfe, M. (1995). Role of the ubiquitin-proteosome pathway in regulating abundance of the cyclin-dependent kinase inhibitor p27. Science 269, 682-685.

Pickering, J. G., Uniyal, S., Ford, C. M., Chau, T., Laurin, M. A., Chow, L. H., Ellis, C. G., Fish, J., and Chan, B. (1997). Fibroblast growth factor-2 potentiates vascular smooth muscle cell migration to platelet-derived growth factor: upregulation of alpha2beta1 integrin and disassembly of actin filaments. Circ Res. 80, 627-37.

Poon, M., Marx, S. O., Gallo, R., Badimon, J. J., Taubman, M. B., and Marks, A. R. (1996). Rapamycin inhibits vascular smooth muscle cell migration. J. Clin. Invest. 98, 2277-2283.

Poston, R. S., Billingham, M., Hoyt, E. G., Pollard, J., Shorthouse, R., Morris, R. E., and Robbins, R. C. (1999). Rapamycin reverses chronic graft vascular disease in a novel cardiac allograft model. Circulation 100, 67-74.

Radeva, G., Petrocelli, T., Behrend, E., Leung-Hagesteijn, C., Filmus, J., Slingerland, J., and Dedhar, S. (1997). Overexpression of the integrin-linked kinase promotes anchorage-independent cell cycle progression. J. Biol. Chem. 272, 13937-13944.

Sabatini, D. M., Erdjument-Bromage, H., Lui, M., Tempst, P., and Snyder, S. H. (1994). RAFT1: A mammalian protein that binds to FKBP12 in a rapamycin-dependent fashion and is homologous to yeast TORs. Cell 78, 35-43.

Schmidt, A., Bickle, M., Beck, T., and Hall, M. N. (1997). The yeast phosphatidylinositol kinase homolog TOR2 activates RHO1 and RHO2 via the exchange factor ROM2. Cell 88, 531-542.

Schwartz, S. M. (1997). Smooth muscle migration in atherosclerosis and restenosis. J. Clin. Invest. 100, S87-98.

Seasholtz, T. M., Majumdar, M., Kaplan, D. D., and Brown, J. H. (1999). Rho and Rho kinase mediate thrombin-stimulated vascular smooth muscle cell DNA synthesis and migration. Circ Res 84, 1186-1193.

Sheaff, R. J., Groudine, M., Gordon, M., Roberts, J. M., and Clurman, B. E. (1997). Cyclin E-CDK2 is a regulator of p27^{kip1}. Genes Dev. 11, 1464-1478.

Shirane, M., Harumiya, Y., Ishida, N., Hirai, A., Miyamoto, C., Hatakeyama, S., Nakayama, K., and Kitagawa, M. (1999). Down-regulation of p27^{kip1} by two mechanisms, ubiquitin-mediated degradation and proteolytic processing. J Biol Chem 274, 13886-13893.

Smith, R. C., Branellec, D., Gorski, D. H., Guo, K., Perlman, H., Dedieu, J. F., Pastore, C., Mahfoudi, A., Denefle, P., Isner, J. M., and Walsh, K. (1997). p21cip1-mediated inhibition of cell proliferation by overexpression of the gax homeodomain gene. Genes Dev. 11, 1674-89.

Sollott, S. J., Cheng, L., Pauly, R. R., Jenkins, G. M., Monticone, R. E., Kuzuya, M., Froehlich, J. P., Crow, M. T., Laketta, E. G., Rowinsky, E. K., and Kinsella, J. L. (1995). Taxol inhibits neointimal smooth muscle cell accumulation after angioplasty in the rat. J. Clin. Invest. 95, 1869-1876.

Sousa, J. E., Costa, M. A., Abizaid, A., Abizaid, A. S., Feres, F., Pinto, I. M. F. et al. (2000) Lack of neointimal proliferation after implantation of sirolimus-coated stents in human coronary arteries. A quantitative coronary angiography and three-dimensional intravascular ultrasound study. Circulation 102, r54-r57.

Spector, D. L., Goldman, R. D., and Leinwand, L. A. (1997). Cells: a laboratory manual. (New York: Cold Spring Harbor Laboratory Press).

Steeg, P. S., and Abrams, J. S. (1997). Cancer prognostics: Past, present and p27. Nature Med. 3, 152-154.

Tanner, F. C., Yang, Z. Y., Duckers, E., Gordon, D., Nabel, G. J., and Nabel, E. G. (1998). Expression of cyclin-dependent kinase inhibitors in vascular disease. Circ Res 82, 396-403.

Vlach, J., Hennecke, S., and Amati, B. (1997). Phosphorylation-dependent degradation of the cyclin-dependent kinase inhibitor p27^{kip1}. EMBO J. 16, 5334-5344.

Wang, W., Chen, H. J., Warshofsky, M., Schwartz, A., C. A., S., and L. E., R. (1997). Effects of S-dC28 on vascular smooth muscle cell adhesion and plasminogen activator production. Antisense & Nucleic Acid Drug Development 7, 101-107.

Weber, J. D., Hu, W., Jefcoat, S. C., Raben, D. M., and Baldassare, J. J. (1997). Ras-stimulated extracellular signal-related kinase 1 and RhoA activities coordinate platelet-derived growth factor-induced G1 progression through the independent regulation of cyclin D1 and p27^{kip1}. J Biol Chem 272, 32966-32971.

Witzenbichler, B., Kureishi, Y., Luo, Z., Le Roux, A., Branellec, D., and Walsh, K. (1999). Regulation of smooth muscle cell migration and integrin expression by the Gax transcription factor. J. Clin. Invest. 104, 1469-1480.

What is claimed is:

1. A method of preventing migration of a cell by increasing intracellular cyclin-dependent kinase inhibitor p27 activity.

2. The method of claim 1, wherein the cell is a smooth muscle cell or a tumor cell.

3. A method of treating a subject's cardiovascular disease, which comprises administering to the subject a compound which increases intracellular cyclin-dependent kinase inhibitor p27 activity, thereby alleviating the subject's cardiovascular disease.

4. The method of claim 3, wherein the cardiovascular disease is atherosclerosis, arteriopathy after heart transplantation, or restenosis after angioplasty or coronary stent placement.

5. A method of inhibiting tumor metastasis in a subject, which comprises administering to the subject a compound which increases intracellular cyclin-dependent kinase inhibitor p27 activity, thereby inhibiting tumor metastasis.

6. The method of claim 1, 3, or 5, wherein cyclin-dependent kinase inhibitor p27 activity is increased by increasing C3 exoenzyme activity.

7. A method of identifying a chemical compound that inhibits cellular migration, which comprises contacting cells whose migration is inhibited when intracellular cyclin-dependent kinase inhibitor p27 activity is increased, or contacting an extract from said cells, with the chemical compound under conditions suitable for increasing p27 activity, and detecting an increase in p27 activity in the presence of the chemical compound so as to thereby identify the chemical compound as a compound which inhibits cellular migration.

8. The method of claim 7, wherein the chemical compound is not previously known to inhibit cellular migration.

9. A method of screening a plurality of chemical compounds not known to inhibit cellular migration to identify a chemical compound which inhibits cellular migration, which comprises:
(a) contacting cells whose migration is inhibited when intracellular cyclin-dependent kinase inhibitor p27 activity is increased, or contacting an extract from said cells, with the plurality of chemical compounds under conditions suitable for increasing p27 activity;
(b) determining if p27 activity is increased in the presence of the plurality of chemical compounds; and if so
(c) separately determining if p27 activity is increased in the presence of each compound included in the plurality of chemical compounds, so as to thereby identify any compound included therein as a compound which inhibits cellular migration.

10. The method of claim 7 or 9, wherein the cells are smooth muscle cells or tumor cells.

11. The method of claim 7 or 9, wherein the cells are vertebrate cells.

12. The method of claim 11, wherein the vertebrate cells are mammalian cells.

13. The method of claim 12, wherein the mammalian cells are human cells.

14. A chemical compound identified by the method of claim 7 or 9.

15. A pharmaceutical composition comprising (a) an amount of a chemical compound identified using the method of claim 7 or 9, or a novel structural and functional homolog or analog thereof, capable of passing through a cell membrane and effective to increase intracellular cyclin-dependent kinase inhibitor p27 activity and (b) a pharmaceutically acceptable carrier capable of passing through the cell membrane.

16. A pharmaceutical composition comprising an amount of a chemical compound identified using the method of claim 7 or 9 effective to inhibit cellular migration and a pharmaceutically acceptable carrier.

17. A method for preparing a composition which comprises admixing a carrier and a pharmaceutically effective amount of a chemical compound identified by the method of claim 7 or 9 or a novel structural and functional analog or homolog thereof.

18. A method for making a composition of matter which inhibits cellular migration which comprises identifying a chemical compound using the method of claim 7 or 9, and then synthesizing the chemical compound or a novel structural and functional analog or homolog thereof.

19. A method of treating a subject with a cardiovascular disease which comprises administering to the subject a therapeutically effective amount of a chemical compound identified by the method of claim 7 or 9, or a novel structural and functional analog or homolog thereof.

20. The method of claim 19, wherein the cardiovascular disease is atherosclerosis, arteriopathy after heart transplantation, or restenosis after angioplasty or coronary stent placement.

21. A method of inhibiting tumor metastasis in a subject which comprises administering to the subject a therapeutically effective amount of a chemical compound identified by the method of claim 7 or 9, or a novel structural and functional analog or homolog thereof.

22. Use of a chemical compound identified by the method of claim 7 or 9 for the preparation of a pharmaceutical composition for treating an abnormality, wherein the abnormality is alleviated by inhibiting cellular migration.

23. The use of claim 22, wherein the abnormality is a cardiovascular disease or a tumor metastasis.

24. The use of claim 23, wherein the cardiovascular disease is atherosclerosis, arteriopathy after heart transplantation, or restenosis after angioplasty or coronary stent placement.
